SEQUENCE LISTING (4 sequences)

SEQ ID NO: 1 - DNA, 20 bp, Artificial Sequence
  Description of Artificial SequencePCR primer
ctctcggagtggccattcga

SEQ ID NO: 2 - DNA, 20 bp, Artificial Sequence
  Description of Artificial SequencePCR primer
ggcacattggaactgagcac

SEQ ID NO: 3 - protein, 30 aa, Artificial Sequence
  Description of Artificial Sequenceamino acids 61-90 of alpha synuclein peptide
EQVTNVGGAVVTGVTAVAQKTVEGAGSIAA

SEQ ID NO: 4 - protein, 15 aa, Artificial Sequence
  Description of Artificial Sequenceamino acids 61-75 of alpha synuclein peptide
EQVTNVGGAVVTGVT

(19) United States
(12) Patent Application Publication
(10) Pub. No.: US 2003/0027210 A1
(43) Pub. Date: Feb. 6, 2003

(54) Alpha synuclein aggregation assays

(76) Inventors: Daniel Benjamin, Barnegat, NJ (US); Sergey Ilyin, Hatboro, PA (US); Carlos R. Plata-Salaman, Ambler, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/037,519

(22) Filed: Jan. 3, 2002

Related U.S. Application Data
(60) Provisional application No. 60/259,442, filed on Jan. 3, 2001.

(51) Int. Cl. G01N 33/53

(52) U.S. Cl. 435/7.1

(57) ABSTRACT

The present invention provides methods to measure alpha synuclein aggregation in vitro. The methods of the present invention are useful to determine the anti-aggregation potential of compounds or to screen for drugs with anti-aggregation or dis-aggregation properties.

### CROSS REFERENCE TO RELATED APPLICATIONS

This application claims priority from U.S. provisional application Serial No. 60/259,442 filed Jan. 3, 2001, the contents of which are hereby incorporated by reference.

### FIELD OF THE INVENTION

The present invention provides methods to measure alpha synuclein aggregation in vitro. The methods of the present invention are useful to determine the anti-aggregation potential of compounds or to screen for drugs with anti-aggregation or dis-aggregating properties.

### BACKGROUND OF THE INVENTION

Alpha Synuclein is a 140 amino acid protein that can aggregate and precipitate into dense intracytoplasmic inclusions known as Lewy bodies, which are involved in the etiology of Lewy body dementia, diffuse Lewy body disease, Alzheimer's disease with Parkinsonism, Lewy body variant of Alzheimer's disease, Parkinson's disease with dementia, and Parkinson's disease.

Sporadic and familial Parkinson's disease exhibit similar clinical manifestations and neuropathological profiles (Lewy bodies and Lewy neurites in the substantia nigra and other brain regions). Aggregated alpha synuclein is the principal component of Lewy bodies and Lewy neurites (Spillantini et al., 1998). In fact, Lewy bodies and Lewy neurites are pathognomonic of Parkinson's disease and Lewy body dementia. Two different point mutations in the alpha synuclein gene (A53T and A30P) were identified in separate families with dominantly transmitted Parkinson's disease (Polymeropoulos et al., 1998 and patent application WO 98/5950, published 12/30/1998). These point mutations of alpha synuclein have been shown to increase the ability of alpha synuclein to aggregate (Narhi et al., 1999; Wood et al., 1999) and to slow down degradation of the mutated alpha synuclein (Bennett et al., 1999). The consistent effect of these mutations in increasing the amount and aggregation of alpha synuclein suggests that these processes play an important role in the pathophysiology of various neurodegenerative disorders. In fact, overexpression of wild-type alpha synuclein is associated with cellular toxicity (Ostrerova et al., 1999). Thus, compounds that inhibit alpha synuclein aggregation represent a novel therapeutic strategy as disease-modifying agents for neurodegeneration.

It is thought that, when alpha synuclein is damaged by system failure in old age or injury, the protein takes on an aberrant shape or conformation that can be impressed upon other synuclein molecules. These molecules then bind to one another and the protein aggregates accumulate and deposit inside the neuron, where they exert oxidative damage as they increase in size. This process first prevents the neuron from performing its necessary role in brain function and as it progresses, eventually kills the neuron. It would be desirable to develop new drugs that specifically prevent the pathological aggregation of alpha synuclein and/or disperse the toxic aggregates.

Alpha synuclein fragment is a constituent of Alzheimer's disease amyloid plaques, hence the alternative name of non-amyloid component (NAC). Another pathophysiological involvement of alpha synuclein relates to the previously unrecognized high incidence of Lewy body dementia. In the absence of neuropathological evidence, Lewy body dementia is frequently misdiagnosed as Alzheimer's disease.

Patent application WO00/18917 by Biere et al and published Apr. 6, 2000 describes alpha synuclein mutants (A30P/A53T—a double mutant of known naturally occurring mutants, E83Q/A90V, H50Y/A53T, and H50T/A53T/A76T). These mutants are tested by centrifugation methods to confirm aggregation.

Patent application WO00/20020 by Masliah and published Apr. 13, 2000 describes methods of screening alpha synuclein anti-aggregating compounds. Metal induced alpha synuclein aggregation is conducted and measured by Thioflavin-S staining.

Patent application WO99/06545 by Wanker et al and published Feb. 11, 1999 describes GST fusion proteins with polyglutamine polypeptides and assays to monitor aggregation of the polyglutamine repeat domains including using Thioflavin T aggregation.

### SUMMARY OF THE INVENTION

The present invention provides methods to detect changes in the state of aggregation of an alpha synuclein by measuring the fluorescence of the fluorescent dye, Thioflavin T.

One embodiment of the present invention provides methods to detect the ability of a compound to promote disaggregation of an alpha synuclein by comparing the degree of Thioflavin T fluorescence in a sample containing a compound to a similar, fully aggregated control.

Another embodiment of the present invention provides methods to test the potential of a compound to prevent aggregation of an alpha synuclein by comparing the degree of Thioflavin T fluorescence in a sample containing the compound to a similar sample that is allowed to fully aggregate.

### BRIEF DESCRIPTION OF THE DRAWING

FIG. 1: Rifampicin Concentration curves with a 2 hours (filled circles) and 5 hours (open circles) incubation time; RFU=Relative Fluorescence Units (calculated as counts/second× Attenuator factor)

FIG. 2: Effect of time on the Disaggregation of alpha synuclein by Rifampicin. Filled circles=5 minutes, Open circles=15 minutes, Filled triangles=30 minutes, Open triangles=60 minutes, Filled squares=120 minutes

### DETAILED DESCRIPTION

This invention comprises an approach for the treatment of Parkinson's disease, multiple system atrophy, Lewy body dementia, diffuse Lewy body disease, Alzheimer's disease with Parkinsonism, Lewy body variant of Alzheimer's disease, Parkinson's disease with dementia, and related disorders involving the dispersion of neurotoxic aggregates of alpha synuclein and the application of technologies designed to identify chemical compounds with ability to disaggregate alpha synuclein aggregates. The approach to identification of drugs with disaggregating properties involves the use of thioflavin T, a compound that fluoresces when associated with aggregated alpha synuclein. Drugs useful for the treatment of the neurodegenerative disorders mentioned above will cause a decrease in fluorescence emitted by the synuclein aggregate/thioflavin T complex and can be read with a standard fluorimeter at high speed and throughput.

In vitro Anti-Aggregation Assay

In one embodiment of the present invention, methods are provided to detect the ability of a compound to promote disaggregation of an aggregated alpha synuclein comprising the steps, in order:

(a) Adding a compound and Thioflavin T to an aggregated alpha synuclein solution, wherein the Thioflavin T will bind to the aggregated synuclein and produce fluorescence at about 485 nm;

(b) Incubating the solution for sufficient time to allow the compound to change the aggregation state of the synuclein; and

(b) Measuring a reduction of fluorescence at about 485 nm as an indication of a reduced aggregation state of the synuclein.

In another embodiment of the present invention, the methods of the present invention may be used to test the potential to prevent aggregation of an alpha synuclein by a compound. The method comprises the steps, in order:

(a) Combining in an aqueous solution a compound, an alpha synuclein, and Thioflavin T;

(b) Incubating the solution for sufficient time to provide an expected alpha synuclein aggregate, wherein the Thioflavin T will bind to the aggregated synuclein and produce a fluorescence at about 485 nm;

(c) Measuring the amount of fluorescence at about 485 nm and comparing the effect of the compound on alpha synuclein aggregate compared to a similar fully aggregated control.

The alpha synuclein of the present invention may be native protein, protein produced within a cell, may be recombinantly produced and purified, or may be fragments of alpha synuclein, preferably synthetic peptides. Particularly preferred for use in the present invention is a synthetic peptide of alpha synuclein comprising about residues 61 to about 90, EQVTNVGGAVVTGVTAVAQKTVEGAGSIAA (SEQ.ID.NO.:3) of the native alpha synuclein protein. In addition, the alpha synuclein may be a combination of several different forms of alpha synuclein. For example, but not by way of limitation, native protein can be combined with a synthetic peptide, or two or more peptides derived from alpha synuclein can be combined. In particular, synthetic peptides may be used to enhance the rate of aggregation of the alpha synuclein. The amount of “enhancing peptide” used is determined by testing the rate of aggregation of alpha synuclein at various concentrations of the peptides using the methods described herein. Two particular peptides that are useful to enhance the rate of aggregation of alpha synuclein are derived from about residues 61 to about 90 and from about 61 to about 75 of the human alpha synuclein have the sequence as follows:

| | |
|---|---|
| (EQVTNVGGAVVTGVTAVAQKTVEGAGSIAA) | (SEQ.ID.NO.:3), or |
| (EQVTNVGGAVVTGVT) | (SEQ.ID.NO.:4). |

Aggregated alpha synuclein is produced by incubating the protein at a temperature from about 0 to about 50° C. in physiologically balanced buffers. The present invention allows use of any buffer that maintains an appropriate pH and salt concentration to allow the beta sheet aggregate to form. A suitable buffer may be tested by incubating alpha synuclein in the presence of Thioflavin T and monitoring an increased fluorescence. A generally preferred buffer is a phosphate buffered saline solution containing about 200 mM KCl in a pH range of about 6.0 to about 8.0.

The term “compound” as used herein refers to an organic molecule that has the potential to change the aggregation state of an alpha synuclein, either by preventing aggregation, or by disrupting aggregated alpha synuclein. For example, but not to limit the scope of the current invention, compounds may include small organic molecules, other synthetic or natural amino acid peptides, proteins, or synthetic or natural nucleic acid sequences, or any chemical derivatives of the aforementioned. Preferred concentrations of compounds used in the methods of the present invention are in the range of about 0.001 to about 500 micromolar, preferably about 0.001 to about 100 micromolar.

Thioflavin T fluorescence is well known in the art. The term “about” refers to an emission wavelength near the emission maxima of 485 nm. An emission spectrum can be obtained using skills well known in the art, for example in “Principles of Fluorescence Spectroscopy” by Lakeowicz, Plenum Press (1983).

The following examples illustrate the present invention without, however, limiting the same thereto.

### EXAMPLE 1

### Cloning and Expression of Soluble Alpha Synuclein

Recombinant human α-synuclein protein expression and a purification system were developed using standard techniques well known in the art. (See for example, Maniatis, T., Fritsch, E. F., Sambrook, J. Molecular Cloning: A Laboratory Manual, Second Edition (Cold Spring Harbor Laboratory, Cold Spring Harbor, N.Y., 1989). α-Synuclein was amplified from a human brain cDNA library (Clontech) and the sequence was confirmed. Primers 5′-CTCTCGGAGTGGCCATTCGA-′3 (SEQ.ID.NO.:1) and 5′-GGCACATTGGAACTGAGCAC-3′ (SEQ.ID.NO.:2) were designed to amplify a fragment of the human alpha-synuclein cDNA. Amplification was performed by using AmpliTaq DNA Polymerase according to the manufacturer's protocol in a final volume of 100 μl; this was subjected to 35 cycles of denaturation at 95° C. for 60 s and annealing-extension at 60° C. for 90 s. The PCR product was purified by the Wizard PCR Preps DNA Purification System (Promega, Madison, Wis.) and ligated into Srf I site of pCR-Script SK(+) vector according to the manufacturer's protocol (pCR-Script Amp SK(+) Cloning Kit, Stratagene Cloning Systems, La Jolla, Calif.). Following transformation of E. coli Epicurian Coli XL1-Blue MRF′ Kan supercompetent cells (Stratagene), separate colonies were selected, grown overnight, and plasmid DNA was purified by the Wizard Plus Minipreps DNA Purification System (Promega). The identity of the insert was confirmed by sequencing the plasmid DNA from individual colonies.

The α-synuclein cDNA was cloned into a pTYB1 bacterial expression vector. Following transformation of ER2566 E. coli competent cells (New England BioLabs) and 6 h induction at 37° C. recombinant protein was isolated under denaturing conditions using Ni-NTA Resin. Under appropriate conditions, recombinant human a-synuclein formed the predicted aggregates as measured by the thioflavin T (TFT) assay, described herein.

### EXAMPLE 2

### Thoifavin T Screening Assay

A fluorescence-based TFT high-throughput screening assay was developed for the a-synuclein aggregation. Thioflavin T absorbs at 450 nm and emits at 485 nm; fluorescence increases 40-fold in the presence of beta-sheet conformation (LeVine and Scholten). Thioflavin T was obtained from Fluka Chemika. Twenty four micrograms (24 μg) of alpha synuclein protein in assay buffer (0.2 M potassium chloride pH=6; 0.075% sodium azide) was added to microvolume multiwell plate holding 10 μL. Then, Thioflavin T (TFT) was added to each well to a final concentration of 20 μM and contents were thoroughly mixed. Alpha-synuclein was allowed to fully aggregated prior to conducting a dissaggregation assay. Degree of aggregation was measured by increase in Thioflavin T fluorescence. Alpha-synuclein aggregation was conducted with or without seeding (using a peptide to enhance the rate of aggregation). The following peptides were found to speed up synuclein aggregation:

| | | |
|---|---|---|
| syn 61-90 | (EQVTNVGGAVVTGVTAVAQKTVEGAGSIAA) | (SEQ.ID.NO.:3) and |
| syn 61-75 | (EQVTNVGGAVVTGVT) | (SEQ.ID.NO.:4). |

Alpha synuclein aggregates through the formation of a beta sheet structure, measured by increased fluorescence of Thioflavin T at 485 nm. This rapid, inexpensive, and homogeneous screening assay exhibits a coefficient variation of 4-8%.

The aggregated alpha synuclein is assayed to screen compounds for antiaggregation properties. 2 μL of a putative antiaggregation compound diluted in 30% DMSO (final concentration of 40 μM compound) is added to the well. The mixture containing the aggregated alpha synuclein/TFT complex and the compound is incubated for 4 hours at room temperature. Compounds that promote disaggregation of the complex are observed by a decrease of fluorescence compared to wells containing alpha synuclein/TFT complex.

### EXAMPLE 3

Methods:

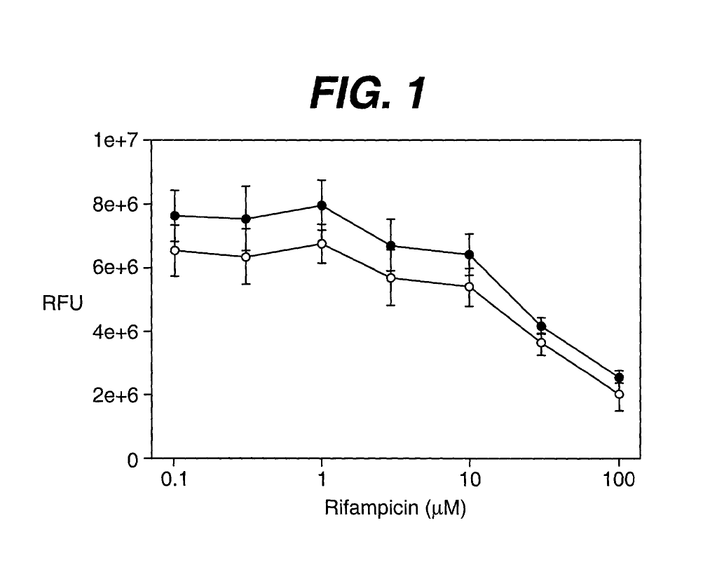

Prior to assay, concentrated alpha synuclein peptide 61-90 (SEQ.ID.No.:3) was diluted into assay buffer containing Thioflavin T (20 uM) such that a signal to noise of approximately 5 to 1 was achieved. Rifampicin was maintained in 8% DMSO at a concentration of 2 mM. To break up super aggregates, the solution was sonicated using a Heat System sonicator with a microprobe. The samples were sonicated in 30 ml volume in a Coming 50 ml centrifuge tube for 25, 3 second bursts. The assay was run in LJL HE (LJL Biosystems) plates as follows: 18 μl of alpha synuclein/thioflavin T mixture was added to the well followed by 2 μl of a 30% DMSO or by rifampicin diluted in 30% DMSO. As a control, buffer with thioflavin T was added to separate wells in the absence of alpha synuclein. The samples were incubated at room temperature for the times indicated in FIG. 1 and were then read on the LJL reader using wavelengths of 440 nm and 485 nm.

Results

Incubation Times:

To test the effect of incubation times on disaggregation of alpha synuclein, alpha synuclein was incubated with a rifampicin concentration curve ranging from 0.1 μM to 100 μM for 2 and 5 hours. The concentration response curves are shown in FIG. 1 and the EC50 values and signal to background are shown in Table 1. The data are the mean of 11 samples±the standard deviation at each time point.

TABLE 1
Effect of Incubation time on alpha synuclein disaggregation

| Incubation time | | Rifampicin EC₅₀ |
|---|---|---|
| (hours) | Signal/Background | (μM) |
| 2 | 4.7 | 23.9 |
| 5 | 4.6 | 21.0 |

The signal to background are from quadruplicate samples of buffer (low value) and alpha synuclein/TFT buffer (high value). The EC50 were generated from 11 samples of each time point.

These data indicate that the 2 hour and 5 hour incubation times were identical with respect to signal to background as well as rifampicin EC50. However, the concentration response curve shown in FIG. 1 indicates that rifampicin only had an effect at concentrations ranging from 1 μM to 100 μM.

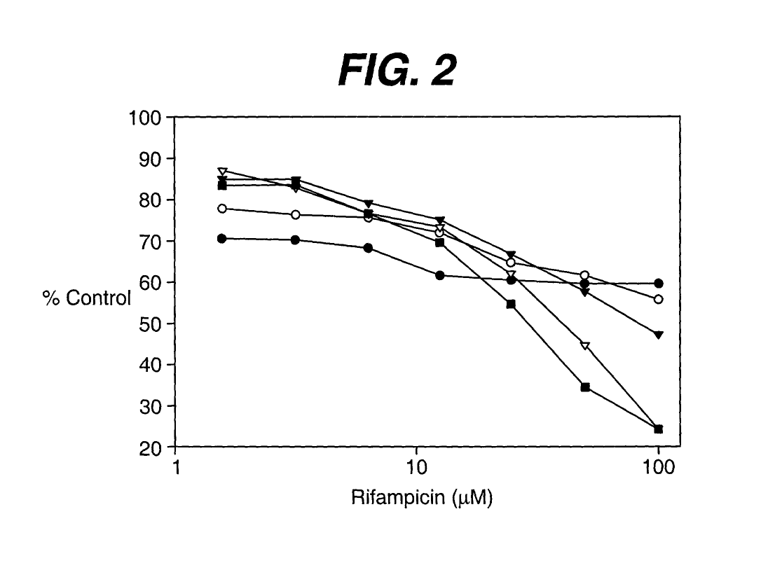

To narrow down the incubation times as well as to identify the concentrations of rifampicin to use, the above study was repeated using incubation times ranging from 5 minutes to 2 hours and rifampicin concentrations of 1.56 μM to 15 μM. The concentration responses are shown in FIG. 2. The EC50 and signal to background are shown in Table 2.

TABLE 2
Signal to Background and EC₅₀ for Rifampicin to Disaggregate
alpha synuclein: Effect of incubation time.

| Incubation time | | EC₅₀ |
|---|---|---|
| (Minutes) | Signal/background | (μM) |
| 5 | 5.3 | — |
| 15 | 6.4 | 311 |
| 30 | 6.7 | 76 |
| 60 | 5.7 | 16.8 |
| 120 | 4.9 | 10.6 |

The data indicate that the rifampicin begins to disaggregate the alpha synuclein sheet by 15 minutes. By 1 hour there is disaggregation that appears complete: there is no further disaggregation with the increased incubation time of 2 hours. The signal to background remained adequate regardless of the incubation time. The EC50 for 60 and 120-minute incubations look identical.

References

Bennett M C, Bishop J F, Leng Y, Chock P B, Chase T N, Mouradian M M. Degradation of alpha-synuclein by proteasome. J Biol Chem 1999, 274:33855-8.

LeVine, H., Scholten, J. D. Screening for pharmacologic inhibitors of amyloid fibril formation. Meth Enzymol 1999, 309(Ch. 29):467-76.

Maroteaux L, Campanelli J T, Scheller R H: a neuron-specific protein localized to the nucleus and presynaptic nerve terminal. J Neurosci 1988 August;8(8):2804-15.

Narhi L, Wood S J, Steavenson S, Jiang Y, Wu G M, Anafi D, Kaufman S A, Martin F, Sitney K, Denis P, Louis J C, Wypych J, Biere A L, Citron M. Both familial Parkinson's disease mutations accelerate alpha-synuclein aggregation. J Biol Chem 1999, 274:9843-6.

Ostrerova N, Petrucelli L, Farrer M, Mehta N, Choi P, Hardy J, Wolozin B. alpha-Synuclein shares physical and functional homology with 14-3-3 proteins. J Neurosci 1999, 19:5782-91.

Polymeropoulos, M H. Autosomal dominant Parkinson's disease and alpha-synuclein. Ann Neurol 1998, 44(3 Suppl 1):S63-4.

Spillantini M G, Crowther R A, Jakes R, Hasegawa M, Goedert M. Alpha-Synuclein in filamentous inclusions of Lewy bodies from Parkinson's disease and dementia with lewy bodies. Proc Natl Acad Sci USA 1998, 95:6469-73.

Wood S J, Wypych J, Steavenson S, Louis J C, Citron M, Biere A L. Alpha-synuclein fibrillogenesis is nucleation-dependent. Implications for the pathogenesis of Parkinson's disease. J Biol Chem 1999, 274:19509-12.

What is claimed is:

1. A method to detect the ability of a compound to promote disaggregation of alpha synuclein comprising the steps, in order:
(a) adding a compound and Thioflavin T to an aggregated alpha synuclein solution, wherein the Thioflavin T will bind to the aggregated synuclein and produce a fluorescence at about 485 nm;
(b) incubating the solution of step (a) for sufficient time to allow the compound to change the aggregation state of the synuclein; and
(c) measuring a reduction of fluorescence at about 485 nm as an indication of a reduced aggregation state of the synuclein.

2. The method of claim 1 wherein the alpha synuclein is a purified, recombinant protein.

3. The method of claim 2 wherein the aggregated alpha synuclein further comprises an enhancing peptide selected from the group consisting of SEQ.ID.NO.:3 and SEQ.ID.NO.:4.

4. The method of claim 1 wherein the alpha synuclein is a synthetic peptide comprising residues 61-90 of alpha synuclein (SEQ.ID.NO.:3).

5. A method to detect the ability of a compound to prevent aggregation of an alpha synuclein comprising the steps, in order:
(a) combining in an aqueous solution a compound, an alpha synuclein, and Thioflavin T;
(b) incubating the solution of step (a) for sufficient time to provide an expected alpha synuclein aggregate, wherein the Thioflavin T will bind to the aggregated synuclein and produce a fluorescence at about 485 nm;
(c) measuring the amount of fluorescence at about 485 nm and comparing the effect of the compound on alpha synuclein aggregate compared to a similar fully aggregated control.

6. The method of claim 5 wherein the alpha synuclein is a purified, recombinant protein.

7. The method of claim 6 wherein the aggregated alpha synuclein further comprises an enhancing peptide selected from the group consisting of SEQ.ID.NO.:3 and SEQ.ID.NO.:4.

8. The method of claim 5 wherein the alpha synuclein is a synthetic peptide comprising residues 61-90 of alpha synuclein (SEQ.ID.NO.:3).
